(19) United States
(12) Patent Application Publication
(10) Pub. No.: US 2016/0321428 A1
(43) Pub. Date: Nov. 3, 2016

(54) Customizable Health Monitoring

(71) Applicant: Google, Inc., Mountain View, CA (US)

(72) Inventor: Jeffrey L. Rogers, Mountain View, CA (US)

(21) Appl. No.: 14/699,181

(22) Filed: Apr. 29, 2015

(51) Int. Cl. G06F 19/00; A61B 5/01; A61B 5/145; A61B 5/024; A61B 5/11; A61B 5/00; A61B 5/021

(52) U.S. Cl. CPC G06F 19/3456; A61B 5/0008; A61B 5/01; A61B 5/021; A61B 5/024; A61B 5/11; A61B 5/14532; G06F 19/3418; G06F 19/3481

(57) ABSTRACT

This document describes customizable health monitoring. The techniques described enable a medical professional to monitor a person's health in their normal course of life, such as prior to each meal, during exercising, while at the office, and so forth. These techniques also enable monitoring that is tailored to that particular person to better understand a suspected problem or a known condition. By so doing, a medical professional can monitor a person over various times and situations, which adds detail and robustness to the data collected. The techniques permit remote tracking and data transfer as well, thereby enabling the health professional to gain the desired information quickly and easily without requiring the patient or the health professional to wait for, or waste time on, an in-person visit.

### BACKGROUND

Health assessments are usually performed at a hospital or medical practitioner's office. Health monitoring at a hospital or office, however, cannot monitor a person during their normal course of life. This can be a serious limitation because a snapshot captured at a hospital or office may not accurately reflect the person's health. This can be due to the testing being of a short duration, infrequent, or due to the testing being in an artificial environment.

Furthermore, conventional health monitoring is often based on demographics and averages, not a particular person. If a person's blood pressure and heart rate are typical for the person's age, for example, the medical practitioner may assume that the person is in good health. If that person is not typical, however, the measured blood pressure and heart rate—while typical for an average person—may represent a significant health change or negative condition for that person. A long-distance runner that would normally have a low heart rate and low blood pressure, for example, can have a negative heart condition developing with even a typical heart rate and blood pressure.

### SUMMARY

This document describes customizable health monitoring. The techniques described enable a medical professional to monitor a person's health in their normal course of life, such as prior to each meal, during exercising, while at the office, and so forth. These techniques also enable monitoring that is tailored by a medical professional to that particular person to better understand a suspected problem or a known condition. By so doing, the medical professional is enabled to monitor a person over various times and situations, which adds detail and robustness to the data collected. The techniques permit remote tracking and data transfer as well, thereby enabling the health professional to gain the desired information quickly and easily without requiring the patient or the health professional to wait for, or waste time on, an in-person visit.

This summary is provided to introduce simplified concepts concerning customizable health monitoring, which is further described below in the Detailed Description. This summary is not intended to identify essential features of the claimed subject matter, nor is it intended for use in determining the scope of the claimed subject matter.

### BRIEF DESCRIPTION OF THE DRAWINGS

Embodiments of techniques and devices for customizable health monitoring are described with reference to the following drawings. The same numbers are used throughout the drawings to reference like features and components:

FIG. 1 illustrates an example environment in which customizable health monitoring can be implemented.

FIG. 2 illustrates an example mobile computing device of FIG. 1.

FIG. 3 illustrates an example health-monitoring device of FIG. 1.

FIG. 4 illustrates an example smart-phone communicating wirelessly with a wireless insulin pump and glucose meter.

FIG. 5 illustrates a method enabling or using customizable health monitoring.

FIG. 6 illustrates a radar-sensed skeletal movement of a patient sensed by a radar field of a smartphone.

FIG. 7 illustrates an example user interface, which prompts a patient to use a modular, rear-camera blood-oxygen monitor.

FIG. 8 illustrates a patient using a wireless in-mouth thermometer responsive to prompting by a manager through a computing bracelet.

FIG. 9 illustrates a method enabling customizable health monitoring based on passive sensors or patient-associated data.

FIG. 10 illustrates an example device embodying, or in which techniques may be implemented that enable use of, customizable health monitoring.

### DETAILED DESCRIPTION

Overview

This document describes techniques using, and devices enabling, customizable health monitoring. Through use of these techniques and devices, detailed, robust, customizable, and real-life data can be collected. Consider, for example, a case where a person has a potential health problem, such as a heart arrhythmia. This possible heart arrhythmia may not be found during a short visit to a medical office. These techniques, however, permit a medical professional to prescribe a heart monitor and an accompanying customized software program for the person's mobile computing device. By so doing, the customized program can prompt the user to use the heart monitor at particular times or responsive to particular stimuli, such as at a same time of day or during a same physiological state each day. If taken during a same physiological state each day, effects caused by substantial differences in physiological parameters throughout the user's day can be avoided, thereby permitting more-accurate monitoring. The customized program can then provide this data to the medical professional, thereby enabling both relatively fast and yet robust health monitoring of the person's heart arrhythmia.

This is but one example of how the techniques and devices enable customizable health monitoring. Other examples are described below. This document now turns to an example environment, after which example health-monitoring devices, methods, a user interface, and an example computing system are described.

Example Environment

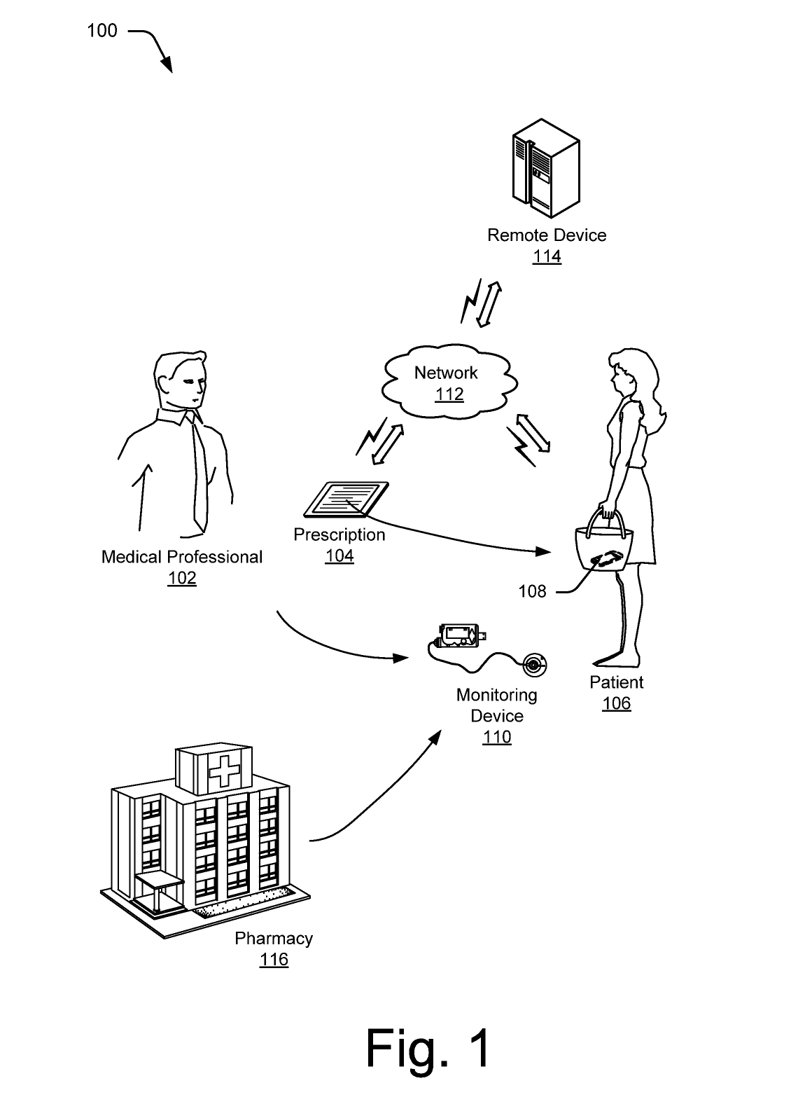

FIG. 1 is an illustration of an example environment 100 in which customizable health monitoring can be employed. Environment 100 illustrates a medical professional 102 prescribing a health-monitoring prescription 104 for a patient 106. This health-monitoring prescription 104 can be determined by medical professional 102 based on patient 106's medical conditions, needs, age, and so forth rather than simply based on a person's demographic or average medical ranges. Further, through use of a mobile computing device 108 and a health-monitoring device 110 (a heart monitor shown as an example), patient 106's medical conditions can be monitored in real life—at various times during the day, after eating, and during exercise, for example, and for extended periods, thereby enabling long-term medical changes to be tracked.

In this environment 100, patient 106 receives prescription 104 either directly from a communication device of medical professional 102 (e.g., a tablet on which prescription 104 is stored) through patient 106's mobile computing device 108 (the smartphone in her purse) or indirectly via communication network 112 and remote device 114. Patient 106 may also receive health-monitoring device 110 directly from medical professional 102 or through another entity, such as brick-and-mortar pharmacy 116. In either case, health-monitoring device 110 is capable of following prescription 104 through use of mobile computing device 108. Note that multiple health-monitoring devices 110 can be prescribed, whether mobile or non-mobile, data from each can be correlated and used to improve accuracy and robustness of measurement results for one various different types of health measurements. Furthermore, prescription 104 may also indicate desired measurements from devices not generally intended for use as health monitors, such as smart phones using accelerometers to measure patient 106's heart rate (when holding the device), walking speed, and so forth.

Network 112 includes one or more of many types of wireless or partly wireless communication networks, such as a local-area-network (LAN), a wireless local-area-network (WLAN), a personal-area-network (PAN), a wide-area-network (WAN), near-field communication (NFC), an intranet, the Internet, a peer-to-peer network, point-to-point network, a mesh network, and so forth.

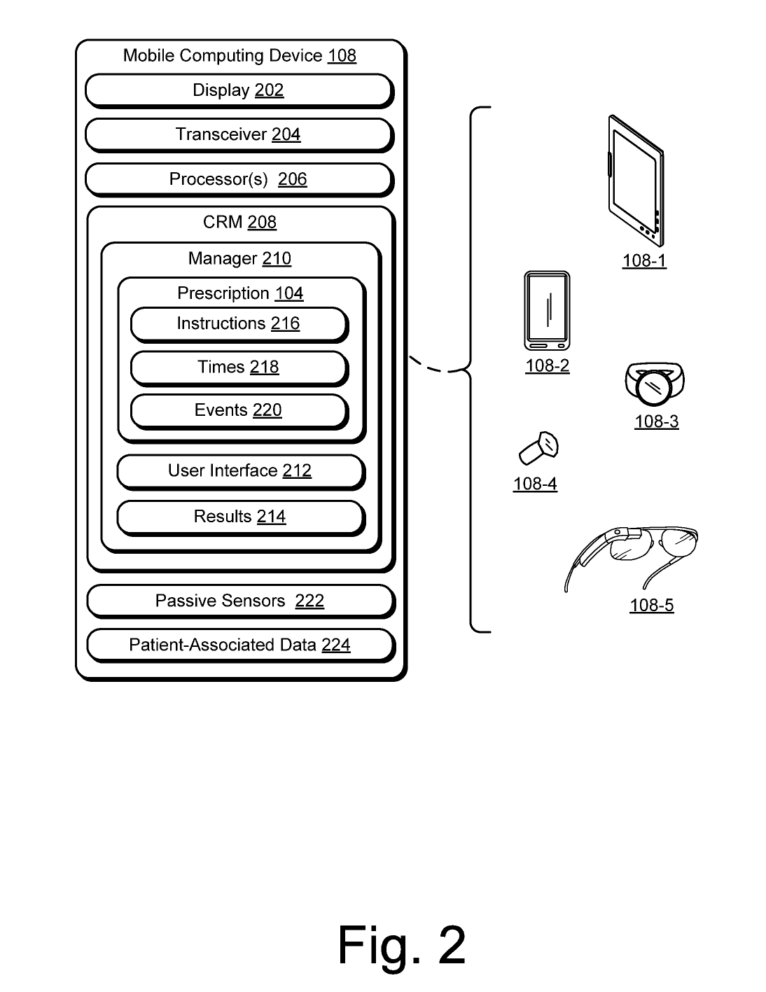

With regard to the example mobile computing device 108 of FIG. 1, consider a detailed illustration in FIG. 2. Mobile computing device 108 can be one or a combination of various devices, here illustrated with five examples: a tablet computer 102-1, a smartphone 102-2, a computing watch 102-3, a computing ring 102-4, and computing spectacles 102-5, though other computing devices and systems, such as a wearable computing device or laptop computer, may also be used. As will be noted in greater detail below, in some embodiments the techniques operate through remote device 114. In such cases, mobile computing device 108 may forgo performing some of the computing operations relating to the techniques, and thus need not be capable of advanced computing operations.

Mobile computing device 108 includes or is able to communicate with a display 202 (five are shown in FIG. 2), a transceiver 204, one or more processors 206, and computer-readable storage media 208 (CRM 208). CRM 208 includes manager 210, which includes or has access to prescription 104, user interface 212, and results 214. Prescription 104 includes instructions 216, times 218, and/or events 220, each of which is described in detail below.

More specifically, prescription 104 may require health-monitoring acts to be made responsive to times 218 or events 220, which can be real-life events, such as the patient eating, sleeping for a period of time, walking, running, or undergoing stress. Further, these real-life events can be sensed by mobile computing device 108 as noted below. Prescription 104's instructions 216 can also include a dynamic adjustment mechanism. This mechanism can indicate, without further instructions from an entity associated with the health-monitoring prescription (e.g., medical professional 102), a different (or changes to) various prescribed health-monitoring acts. These differences may include altering times 218 or events 220 responsive to previously set thresholds for a result of one of the monitoring acts. Thus, if patient 106's blood sugar is monitored at particular events 220 and the results are initially worse or better than expected (e.g., outside of the thresholds), instructions 216 may increase or decrease the number of tests to greater or fewer times or events during patient 106's day.

In some cases prescription 104, through instructions 216, may include a dynamic warning mechanism set by medical professional 102, which can also be acted upon without further instruction from medical professional 102. This permits a flexible and immediate responsiveness to medical changes that is sorely lacking in the current system of a person needing to visit an emergency room at a hospital, set up a doctor's appointment, or hope that a qualified person can be reached in other manners. Results that can trigger this dynamic warning mechanism include acute health conditions, such as dangerously high or low blood sugar, blood pressure, heart rate, heart irregularity, seizures, loss of consciousness, and so forth.

Generally, manager 210 is capable of prompting a patient to initiated use of a health-monitoring device based on prescribed times 218 or events 220 in the prescription. Manager 210 may also or instead cause the health-monitoring device to perform the prescribed monitoring acts and then receive, store, and transmit the results. Manager 210 may wait to transmit the results until the prescribed instructions (instructions 216) are complete, or may transmit results 214 if a particular condition, such as a medical problem needing immediate care, is determined by manager 210.

Mobile computing device 108 may also include or have access to passive sensors 222 and patient-associated data 224, either of which can aid in determining when one of events 220 is occurring. Passive sensors 222 can include an accelerometer that measures movement of mobile computing device 108, and thus implicitly movement of patient 106, a touch sensor of a display screen capable of measuring patient 106's skin temperature, capacitance, and/or conductivity, barometric sensors, light sensors, microphones, and radar sensors capable of passively sensing patient 106's skin temperature, skeletal movement, and heart rate, to name but a few.

In the case of radar sensors, a microwave radio element can be used that provides a radar field configured to reflect from human tissue and penetrate non-human material, such as through continuously modulated radiation, ultra-wideband radiation, or sub-millimeter-frequency radiation. These reflections can be received by an antenna element and signal processor configured to process the reflections from the human tissue in the radar field sufficient to provide data usable to determine a condition of patient 106. This radar field can reflect from human tissue, such as skin, bone, or heart muscle. With these reflections, skin temperature, heart rate, and skeletal movement can be measured, to mention just three examples.

Thus, these passive sensors 222 can passively sense a physical condition of the patient and, based on their data, manager 210 may cause a health-monitoring device to perform an actively sensed physical condition requiring action by the patient. Consider, for example, a case where passive sensors 222 include a radar sensor capable of sensing patient 106's heart rate. Assume that the data indicates that patient 106's heart rate is elevated, and that medical professional 102 indicated, through instructions 216 of prescription 104, that a more-accurate and robust heart monitoring is desired at this heart rate. Manager 210 then prompts patient 106 to attach a device capable of accurate and robust measurement to her chest to measure the heart in detail. Thus, sensors of mobile computing device 108 can passively monitor patient 106 and then, based on this monitoring, active (and presumably superior) sensing can be performed by health-monitoring device 110. Other examples include determining patient 106's movement with an accelerometer, skin temperature with a touch-sensitive display, and so forth, with health-monitoring device 110 capable of active monitoring, such as with an in-mouth thermometer and so forth.

Patient-associated data 224 includes data about patient 106, such as a global position through GPS, cellular, and/or local-area networks (LANs), thereby indicating that patient 106 is out to dinner, walking along a street, driving, at work, at home, and so forth. Patient-associated data 224 may also include patient 106's calendar or other personal information, and thus activities for various times of patient 106's day. Prescribed patient conditions can be determined based on this data and/or sensors, such as patient 106 being at work, at high activity, asleep, or in a particular location. Thus, manager 210 is capable of determining, through use of passive sensors 222 or patient-associated data 224, that a prescribed patient condition is occurring (e.g., one of events 220). Once determined, manager 210 prompts the patient or causes health-monitoring device 110 to monitor patient 106.

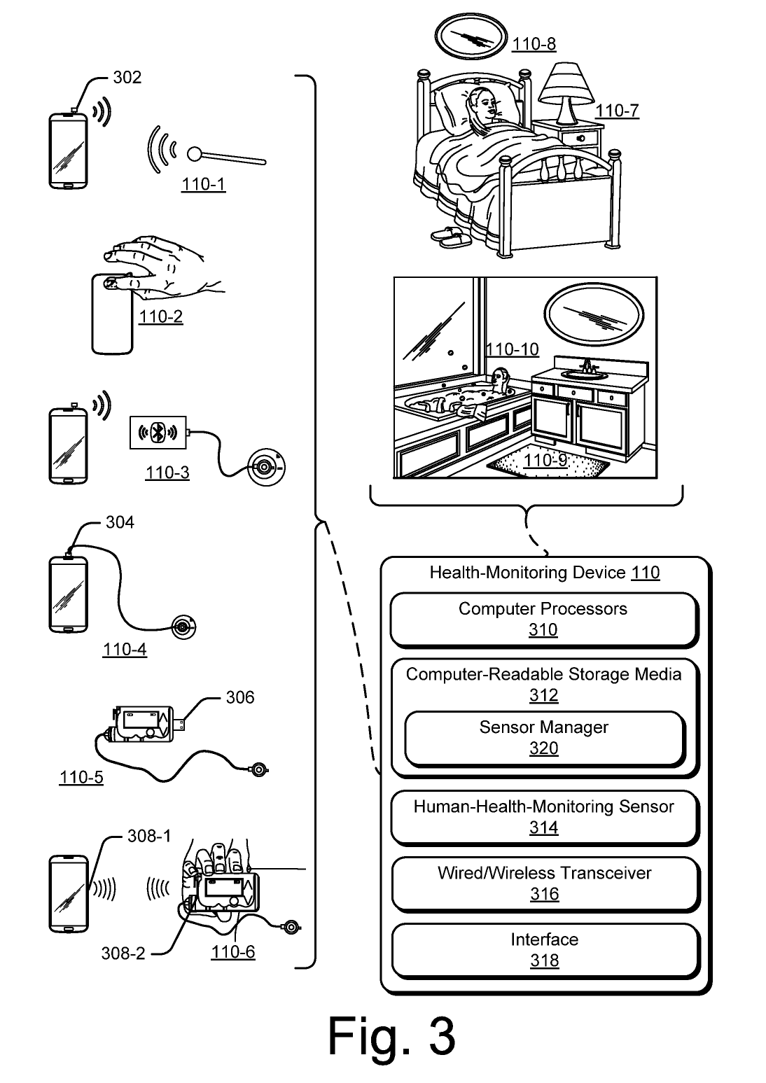

Consider, in more detail, health-monitoring device 110, examples of which are illustrated in FIG. 3. Health-monitoring device 110 can include many different devices, such as those independent of, integral with, separate but in communication with, or modularly integrated with mobile computing device 108. These health-monitoring devices 110 are illustrated with six mobile examples: a wireless in-mouth thermometer 110-1, an integral, modular rear-camera blood-oxygen monitor 110-2, a wireless heart monitor 110-3 (here shown configured to use a personal-area network wireless protocol), a wired heart monitor 110-4, a wired insulin pump and glucose meter 110-5, and a wireless insulin pump and glucose meter 110-6 and four less-mobile examples: a radar-based health-monitoring lamp 110-7, a color-sensing mirror 110-8, pressure and electrical-sensing mat 110-9, and ultrasonic bathtub 110-10.

In more detail, radar lamp 110-7 and/or color-sensing mirror 110-8 are configured to reflect radiation from human tissue to measure skin temperature and perspiration, heart rate, and skeletal movement, to name just three examples. Pressure and electrical-sensing mat 110-9 is configured to sense a pulse-wave velocity of patient 106's blood. This pulse-wave velocity can be used to determine a pressure-volume loop for the patient's heart. This pulse-wave velocity is a measure of a patient's cardiovascular health. In healthy arteries of the cardiovascular system the pulse-wave velocity is low due to the elasticity of the arteries but, as they harden and narrow, the pulse-wave velocity rises. While a particular pulse-wave velocity is a snap shot in time that may or may not accurately indicate cardiovascular health (e.g., a one-time test at a doctor's office), a change in this pulse-wave velocity (that is, a trend), can be an accurate measure of a change in patient 106's cardiovascular health. Ultrasonic bathtub 110-10 is configured to generate high-frequency sound waves and to evaluate an echo from those waves. This echo is received at one or more sensors and the time interval between sending and receiving can be measured. These echoes enable analysis of internal body structures. In some cases, acoustic impedance of a two-dimensional cross-section of tissue can be measured, which can measure current heath or a health trend of the measured tissue. Blood flow, tissue movement, blood location, and three-dimensional measurements of structures can also be made. Non-active (no sound waves generated, just receiving sensors) can also be used, though accuracy and robust measurements are more difficult to achieve.

Other health-monitoring devices are also contemplated herein, such as blood pressure monitors, blood-oxygen monitors, carbon-dioxide monitors (breath or blood), alcohol monitors (breath or blood), brain-activity monitors, seizure monitors (muscle or brain function), body mass and surface area monitors, legal or illegal drug usage (breath, blood, brain activity, or skin), and so forth.

Note that two of these wireless communications are enabled through a wireless transceiver intended for use with, or that is functionally integral with, the health-monitoring device, namely devices 110-1 and 110-3, shown at transceiver 302. Transceiver 302, in some embodiments, attaches to a port (e.g., mini-USB) or audio jack. Transceiver 302 enables communication between health-monitoring device 110 and elements of mobile computing device 108, such as manager 210. In some cases, however, an existing wired or wireless port is used, as illustrated with mini-USB (universal serial bus) connector 304 for wired heart monitor 110-4, standard USB connector 306 for wired insulin pump and glucose meter 110-5, and a wireless near-field communication (NFC) or personal- or local-area network (PAN or LAN) communication system 308 (shown at ports 308-1 and 308-2) for wireless insulin pump and glucose meter 110-6.

Health-monitoring device 110 may having various computing capabilities, though it may instead be a low-capability device having little or no computing capability. Here health-monitoring device 110 includes one or more computer processors 310, computer-readable storage media 312, a human-health-monitoring sensor 314, a wired or wireless transceiver 316 capable of receiving and transmitting information to a mobile computing device associated with a patient, and, in some cases, an interface 318 (e.g., a display or even simple LED indicators). The wired or wireless transceiver 316 includes one or more of the many communication systems noted above. The human-health monitoring sensor 314 may include one of the many monitors described herein (blood oxygen, heart rate, temperature, etc.).

CRM 312 includes sensor manager 320, which is capable of receiving instructions or commands regarding a health-monitoring act or performing a health-monitoring act responsive to a user's interaction (e.g., the user is prompted rather than health-monitoring device 110). Responsive to interaction or instruction, sensor manager 320 causes human-health-monitoring sensor 314 to perform the health-monitoring act and then provides the result (e.g., to mobile computing device 108).

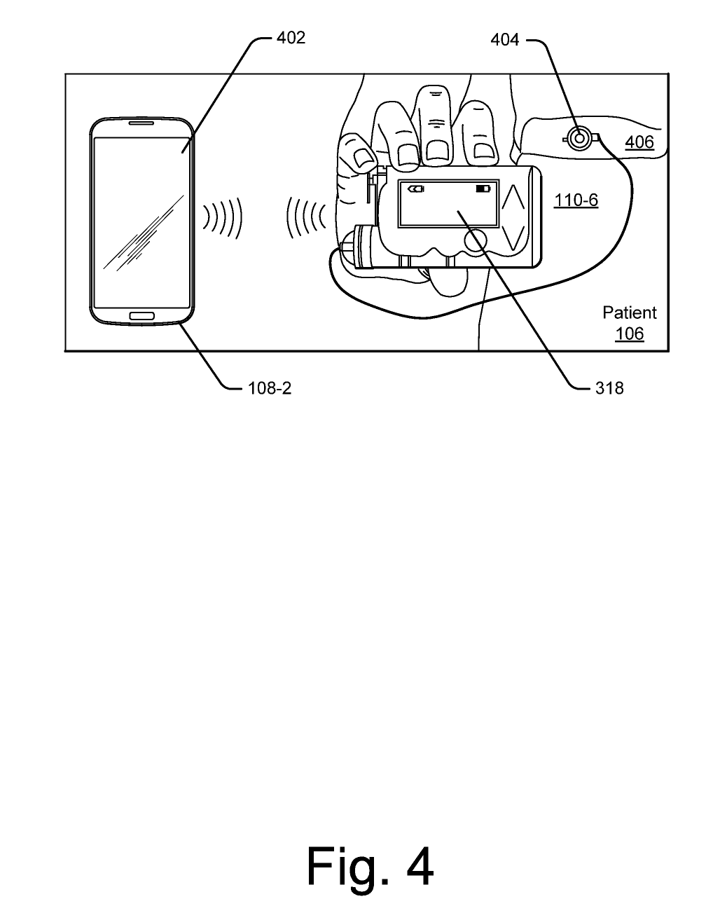

By way of further example, assume that the health-monitoring act prescribed in health-monitoring prescription 104 includes a glucose content for patient 106's blood, and instructions 216 indicate to turn on interface 318 of health-monitoring device 110. Sensor manager 320 turns on interface 318, which provides usage instructions to patient 106, tests patient 106's blood glucose in part with patient 106's help through human-health-monitoring sensor 314, and provides the result to manager 210 of mobile computing device 108. This is illustrated in FIG. 4, which shows smart-phone 108-2 communicating wirelessly with wireless insulin pump and glucose meter 110-6. Sensor manager 320 turns on interface 318 of meter 110-6, though manager 210 may also or instead use a user interface 402 of smartphone 108-2, which can be tailored to how to use meter 110-6 or be somewhat generic to provide usage instructions common to these types of health-monitoring devices. Sensor 404 is also shown, which is attached to patient 106's abdomen 406.

These and other capabilities, as well as ways in which entities of FIGS. 1-4 act and interact, are set forth in greater detail below. These entities may be further divided, combined, and so on. The environment 100 of FIG. 1 and the detailed illustrations of FIGS. 2-4 illustrate some of many possible environments capable of employing the described techniques.

Example Methods

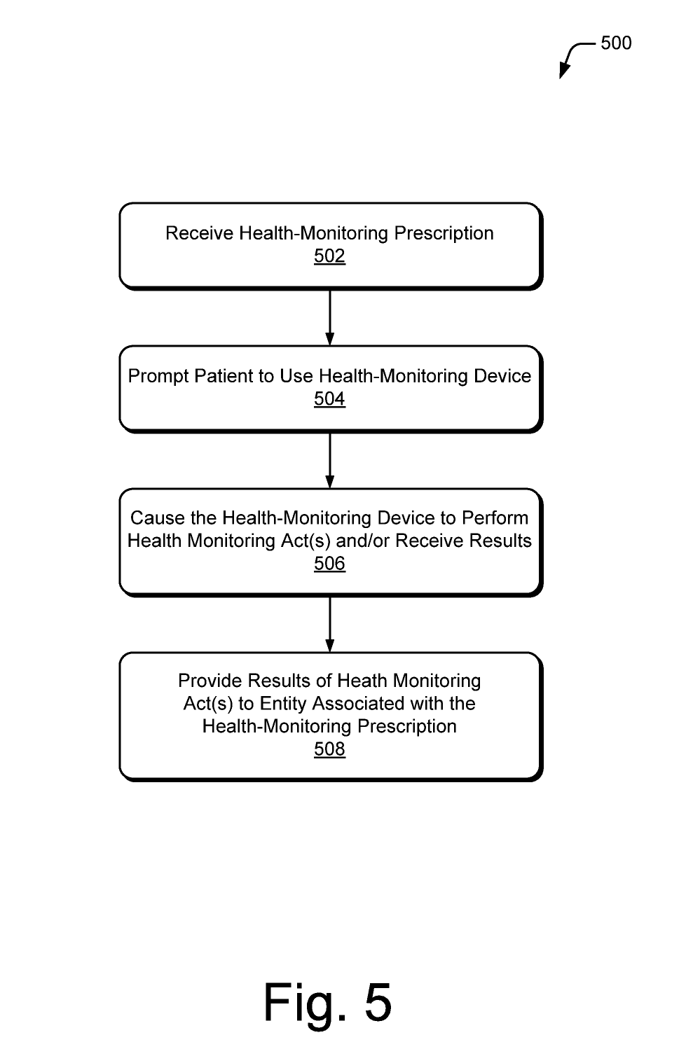

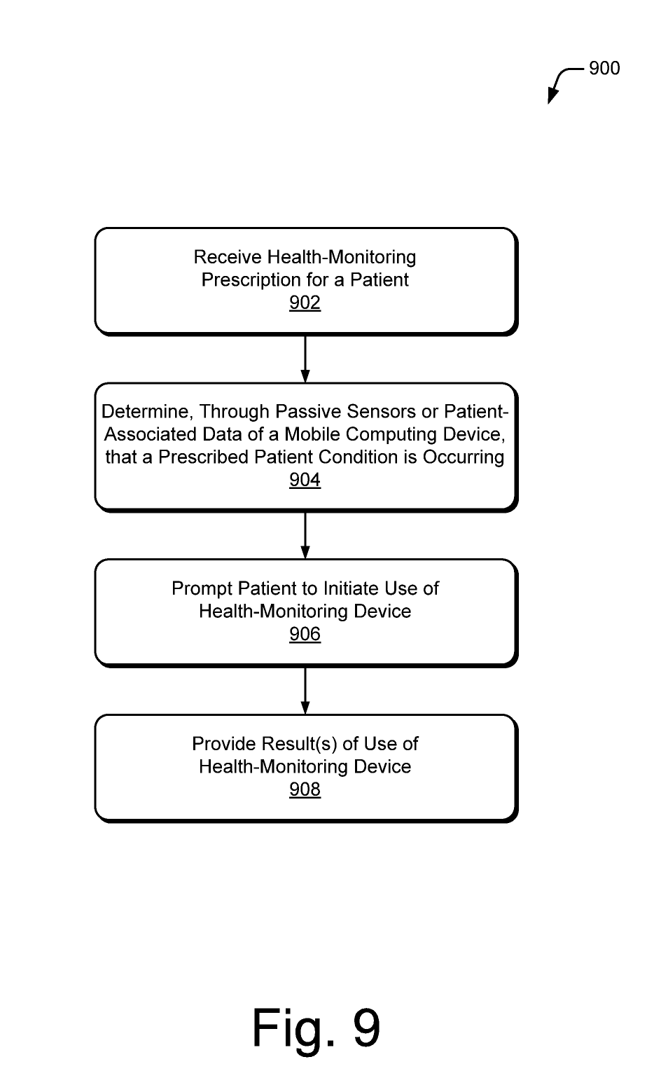

FIGS. 5 and 9 depict methods enabling or using customizable health monitoring. These methods are shown as sets of blocks that specify operations performed but are not necessarily limited to the order or combinations shown for performing the operations by the respective blocks. In portions of the following discussion reference may be made to environment 100 of FIG. 1 and entities detailed in FIGS. 2 and 3, reference to which is made for example only. The techniques are not limited to performance by one entity or multiple entities operating on one device.

At 502, a health-monitoring prescription for a patient is received. As noted, this prescription can be created by a health professional, such as a medical doctor, physical therapist, mental-health professional, registered nurse practitioner, or, in some cases, be prescribed from non-medical persons or the patient himself, such as for advanced health monitoring for improving and tracking fitness and so forth. As noted, this prescription may include events or times at which to monitor a patient's health.

By way of example, assume that a surgeon performs surgery on a patient and wants to carefully track the patient's health. Open wounds common to surgery often fester, fail to close, or otherwise fail to heal. This can be due to local chemistry at the open wound, as one example. This local chemistry can be determined with a sensor (e.g., some type of health-monitoring device 110), which is capable of determining oxygen or other gas contents at the open wound. These gases correlate to how well the open wound is healing or likely to heal. Other examples include cell movement, bacteria content, and so forth as well, which can also be sensed, thereby quickly indicating a negative condition, and therefore permitting a medical professional to quickly address the negative condition. Assume that the surgeon prescribes this type of health-monitoring device 110 along with a schedule set to times and/or events at which to place a sensor at the open wound.

At 504, responsive to receiving the health-monitoring prescription and based on the prescribed times or events occurring, the patient is prompted to initiate use of a health-monitoring device. In some cases, the techniques prompt the patient by instructing the health-monitoring device to prompt the patient. Thus, manager 210 of mobile computing device 108 may prompt patient 106 directly or through health-monitoring device 110. When prompting, manager 210 may present a chime, song, vibration, visual indicator (through a display interface on the mobile computing device or otherwise, such as a blinking light on health-monitoring device 110), or other manner known for prompting a user of a mobile device.

Continuing the ongoing example, manager 210 prompts the surgeon's patient to place a sensor on, at, or near the open wound noted above to monitor gas content, bacteria, or some other prescribed monitoring.

At 506, the health-monitoring device is caused to perform a health-monitoring act and/or results are received from the health-monitoring device. As noted, these results can be from performance of one or multiple health-monitoring acts for the patient. In some cases health-monitoring device 110 is capable of different monitoring acts or different manners in which to perform them (e.g., two sensors or one sensor applied in two ways). In such cases, manager 210 can cause the health-monitoring device to perform the different monitoring acts or different manners, such as to test for heart rate or blood pressure for a device that can test both.

Continuing the ongoing example, assume that the surgeon's patient, responsive to the prompting, places the sensor at the open wound. The health-monitoring device 110 measures a particular condition (e.g., a gas or bacteria content), and passes this result to mobile computing device 108 and therefore to manager 210.

At 508, responsive to receiving the results from the performance of the health-monitoring acts, the results are provided to an entity associated with the health-monitoring prescription, such as medical professional 102 of FIG. 1. In some cases each result can be provided, while in others a certain number or threshold of certain types of results are recorded prior to providing them. Thus, manager 210 may record results until a threshold of 10 blood pressure readings during elevated activity levels, 10 after waking up, and 30 right after meals, for example. These thresholds can be set in instructions 216 of prescription 104 by medical professional 102.

Concluding the ongoing example, assume various results are received over various times or events, and then passed to the surgeon. These results can be passed one-by-one or in groups. If a particular result indicates (based on instructions in the surgeon's prescription) that a negative health condition is present, manager 210 may alert the surgeon rather than wait until some set of prescribed number, events, or days of tests are performed.

While manager 210 may manage how and when health-monitoring device 110 operates, this management can be fairly passive or fully active. Thus, manager 210 may prompt patient 106 to use the health-monitoring device and then receive an indication from the health-monitoring device that the patient has initiated use of the device prior to causing the device to perform the monitoring. Or manager 210 may simply prompt the patient to use the monitoring device and then passively wait for results.

Furthermore, manager 210 may determine that an event has occurred, or that a result of a monitoring act requires another test. By way of example, assume that prescription 104 includes instructions requiring a blood oxygen test responsive to an elevated heart rate of 120 or more beats per minute or vigorous movement by patient 106.

Assume, for the first scenario of 120 beats per minute, that the elevated heart rate is one of events 220 and, responsive to determining that this event has occurred, manager 210 prompts patient 106 to use modular, rear-camera blood-oxygen monitor 110-2. Thus, at operation 504, manager 210 prompts responsive to determining that patient 106 has a heart rate of 120 or more. Manager 210 may determine this using passive sensors 222, such as through patient 106's heart rate through her thumb while on display 202 of tablet computer 108-1, or through an accelerometer of computing bracelet 108-3 or ring 108-4. Once determined, manager 210 prompts patient 106 to test her blood oxygen level.

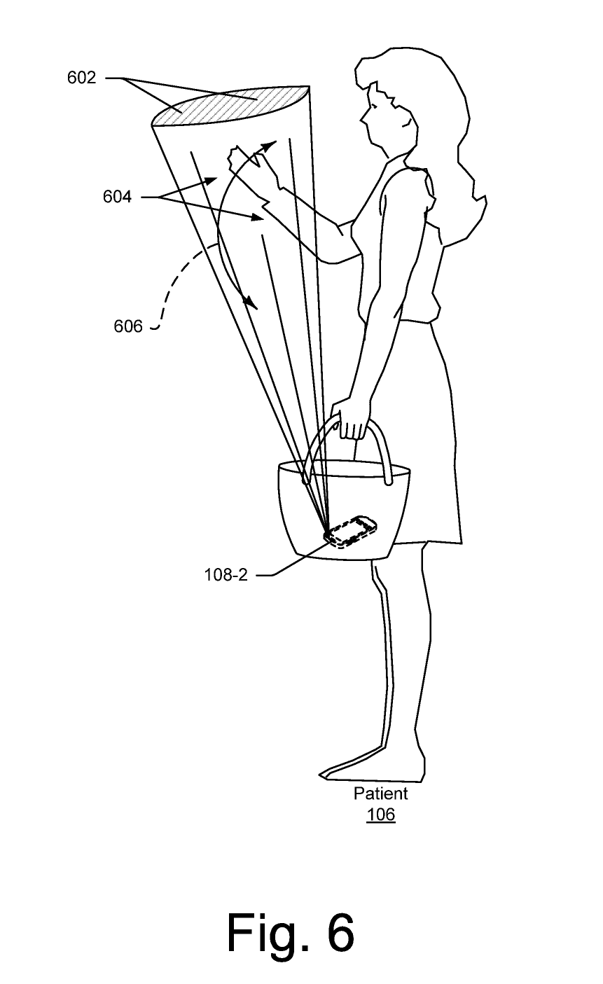

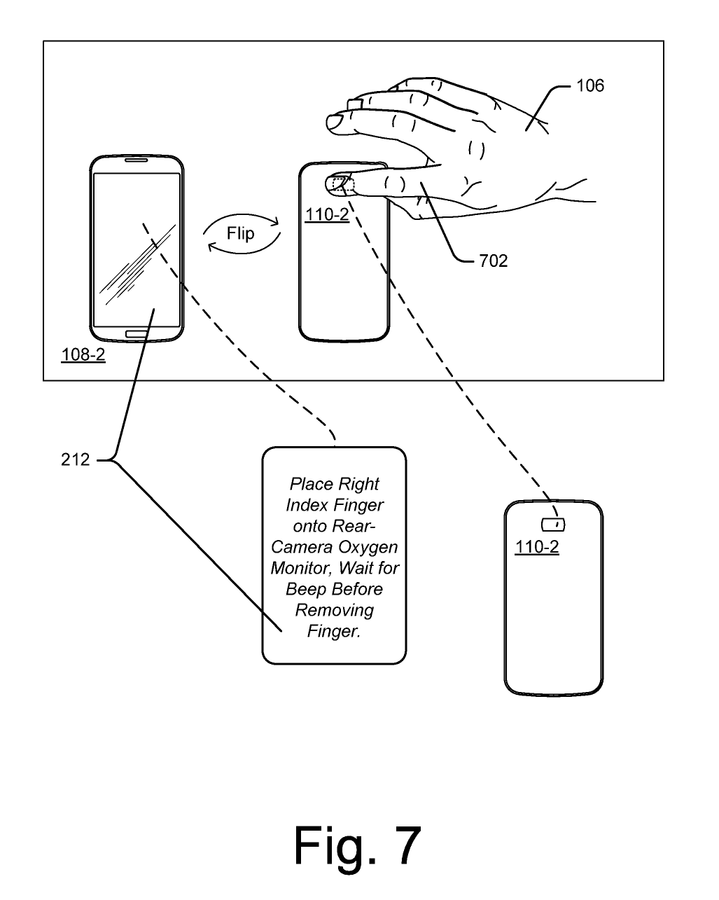

Assume, for the second scenario of vigorous activity, that sustained movement of smartphone 108-2 or radar-sensed skeletal movement of patient 106 sensed by a radar field of smartphone 108-2 are both events indicative of vigorous activity. This is illustrated in FIG. 6, which shows a radar field 602 measuring patient 106's skeletal movement (here arm 604 moving up and down repeatedly, shown at arrow 606). With these and various other passive sensing, manager 210 is able to determine that an event has occurred, often with little or no interference with—or activity required from—patient 106. With this determination made, manager 210 prompts patient 106 to check her blood oxygen level, which is illustrated in FIG. 7.

FIG. 7 shows an example of user interface 212 of FIG. 2, which prompts patient 106 to use modular, rear-camera blood-oxygen monitor 110-2. This user interface 212 flashes, beeps, and presents the following text: “Place Right Index Finger onto Rear-Camera Oxygen Monitor, Wait for Beep Before Removing Finger”. At this point patient 106 simply places her finger over modular, rear-camera blood-oxygen monitor 110-2, waits for a beep, and then when done, moves on with her day without further interruption (unless another time or event in prescription 104 occurs). As the text is shown in user interface 212 at a front of smart-phone 108-2, patient 106 may flip the smart-phone, or simply feel where modular, rear-camera oxygen monitor 110-2 is located to place her finger.

Various health monitors can be modular with removable elements of mobile computing devices, such as the above example. Consider a case where a mobile computing device has a removable speaker unit, microphone unit, or camera unit. In any of these three examples, a health-monitoring device may replace the removable unit. A removable microphone unit can be replaced with various different devices, such as an audio heart-rate or respiration rate monitor tuned to audio associated with these sounds caused by heart beats or respiration or both. A removable speaker unit may be replaced with a health monitor that emits sound for location determination or skin or other organ displacement (e.g., SODAR). In the example of FIG. 7 above, a rear-camera unit is removed and a blood oxygen health monitor replaces it. In this example similar circuitry can be used by the health monitor, as blood oxygen sensors can emit light (e.g., like a camera flash) and, like many cameras, sense light, here reflected from within patient 106's finger 702.

Note that each of these modular health-monitoring devices may have a same or similar form factor as the replaced unit, which has commercial advantages due to a device not being immediately identifiable to other persons. This aids in maintaining patient 106's privacy, as well as allowing standard covers and cases. While these example health-monitoring devices have similarities to the units being replaced, this is not required. A SODAR device may replace a rear camera unit or a radar-enabled sensor a speaker unit, for example.

By way of another example, consider a case where a wearable computing device (e.g., computing bracelet 108-3) has a removable, modular camera. This camera can be removed from its slot and replaced by a medical testing device in that same slot (e.g., blood oxygen sensor, medical-use customized camera, heart-rate monitor, blood-pressure monitor). This medical testing device can be small or even identical in form factor to the removed modular camera. This enables a medical professional to prescribe a particular device for use by the patient and have the patient, without more effort or thought that they usually use in keeping track of their wearable computing device, to have a medical device that is easily accessible, comfortable to wear or carry, and not likely to be lost or misplaced. The device can also take advantage of the computing device's computing power to aid the patient in remembering to use and track the desired medical readings.

Use of modular health-monitoring devices is not limited to use with mobile computers having removable units, as a medical device that is small and can fit well into an existing slot could also be used. Examples include small medical devices that plug into a mini-USB slot or audio jack.

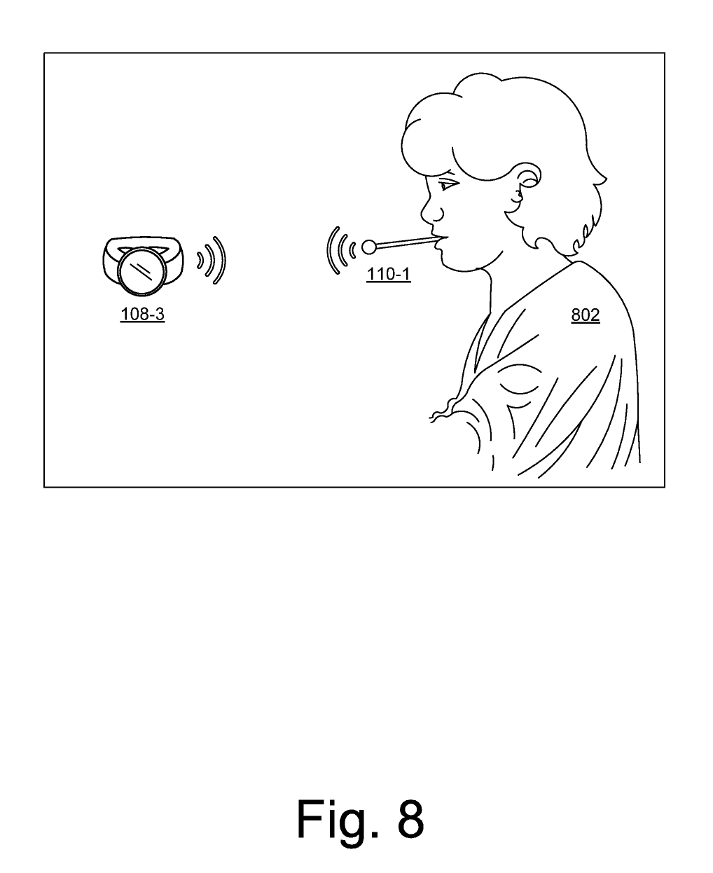

Returning to operation 508 of method 500, note that instructions 216 may indicate, for a particular health-monitoring result, that a more-complex or more-invasive health-monitoring act that tests the same or a similar health condition should be performed. In such a case, manager 210 may prompt a patient to use one of health-monitoring devices 110 to do so. Consider, for example, a situation where passive sensors 222 sense a patient's skin temperature (e.g., through radar, touch display, or backside of a computing ring or bracelet). Based on this skin temperature indicating an elevated temperature (one of events 220 of FIG. 2), instructions 216 indicate that a more-invasive and thus generally more-accurate temperature measurement should be performed. Manager 210 may prompt the user as noted above, and indicate that wireless in-mouth thermometer 110-1 should be used. This is illustrated in FIG. 8 with patient 802 using wireless in-mouth thermometer 110-1 responsive to prompting by manager 210 through computing bracelet 108-3. Note that this permits, in many cases, health monitoring with fewer interruptions to, and activity needed by, patients. A passive sensing of a health condition can be made and, if that condition indicates a potential problem or non-typical result, a monitoring act can be performed that requires some action by a patient. This is preferred, in many cases, as fewer active monitoring sessions are used.

FIG. 9 depicts method 900, which describes manners in which customizable health monitoring is used based on passive sensors or patient-associated data.

At 902, a health-monitoring prescription for a patient is received. This prescription requires multiple health-monitoring acts at prescribed patient conditions. These conditions are types or examples of events 220, described above. These prescribed patient conditions may include a patient working, sleeping, exercising, or being in a particular location, to name just a few.

At 904, the prescribed patient condition is determined to be occurring. This determining can be through one or more passive sensors of a mobile computing device or patient-associated data of the mobile computing device. Various examples of use of passive sensors are set forth above, though with various events including the patient conditions, such as a patient exercising or having a high heart rate.

Patient-associated data 224, as noted above, includes information about the patient that can be used to determine a condition or event for the patient. Thus, manager 210 may determine, based on a calendar, to-do list, social networking site, GPS tracking, and so forth that a user associated with the computing device or directed with the information, is at work, at home, in a park, out to dinner, at a movie, and so forth. Each of these conditions are in real-life and can be used to prompt a health-monitoring act.

At 906, the patient is prompted to initiate use of a health-monitoring device. This is generally responsive to the determination of the condition as noted above. Note that while some health-monitoring is prompted, the prescription may include other health-monitoring acts for which the patient is not prompted. As noted, these other health-monitoring acts can be performed by various devices, including mobile or non-mobile health-monitoring devices of FIG. 3 or passive sensors, such as those of mobile computing devices of 108 of FIG. 2 or in many cases those of mobile-monitoring devices 110-7, 110-8, 110-9, or 110-10 of FIG. 3.

At 908, the result of the health-monitoring act is provided, such as to a medical professional associated with a health-monitoring prescription in which the condition is include as an event at which monitoring is desired. This result is received from the health-monitoring device 110 in the manners noted above.

As is readily apparent, the techniques permit varied and robust health monitoring during a patient's real life, whether at particular times, events, or in particular conditions. In contrast with a medical office, hospital, or other institution, health monitoring through the techniques can be more accurate, more extensive, less intrusive, or simply provide previously unknown health monitoring, such as long-term tracking. Further, this monitoring can be dynamic and responsive, as well as be provided without requiring another visit to the medical professional.

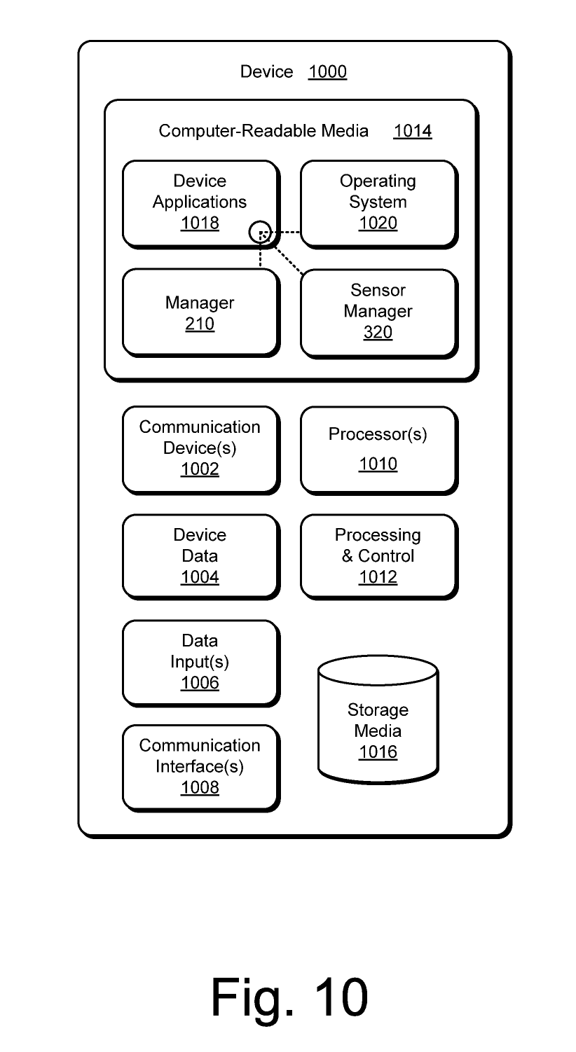

The preceding discussion describes methods relating to customizable health monitoring. Aspects of these methods may be implemented in hardware (e.g., fixed logic circuitry), firmware, software, manual processing, or any combination thereof. These techniques may be embodied on one or more of the entities shown in FIGS. 1-9 and 10 (computing system 1000 is described in FIG. 10 below), which may be further divided, combined, and so on. Thus, these figures illustrate some of the many possible systems or apparatuses capable of employing the described techniques. The entities of these FIGS. generally represent software, firmware, hardware, whole devices or networks, or a combination thereof.

Example Computing System

FIG. 10 illustrates various components of example computing system 1000 that can be implemented as any type of client, server, and/or computing device as described with reference to the previous FIGS. 1-9 to implement customizable health monitoring. In embodiments, computing system 1000 can be implemented as one or a combination of a wired and/or wireless wearable device, System-on-Chip (SoC), and/or as another type of device or portion thereof. Computing system 1000 may also be associated with a user (e.g., a patient) and/or an entity that operates the device such that a device describes logical devices that include users, software, firmware, and/or a combination of devices.

Computing system 1000 includes communication devices 1002 that enable wired and/or wireless communication of device data 1004 (e.g., received data, data that is being received, data scheduled for broadcast, data packets of the data, etc.). Device data 1004 or other device content can include configuration settings of the device, media content stored on the device, and/or information associated with a user of the device. Media content stored on computing system 1000 can include any type of audio, video, and/or image data, including complex or detailed results of human-health-monitoring acts. Computing system 1000 includes one or more data inputs 1006 via which any type of data, media content, and/or inputs can be received, such as human utterances, user-selectable inputs (explicit or implicit), messages, music, television media content, recorded video content, and any other type of audio, video, and/or image data received from any content and/or data source.

Computing system 1000 also includes communication interfaces 1008, which can be implemented as any one or more of a serial and/or parallel interface, a wireless interface, any type of network interface, a modem, and as any other type of communication interface. Communication interfaces 1008 provide a connection and/or communication links between computing system 1000 and a communication network by which other electronic, computing, and communication devices communicate data with computing system 1000.

Computing system 1000 includes one or more processors 1010 (e.g., any of microprocessors, controllers, and the like), which process various computer-executable instructions to control the operation of computing system 1000 and to enable techniques for, or in which can be embodied, customizable health monitoring. Alternatively or in addition, computing system 1000 can be implemented with any one or combination of hardware, firmware, or fixed logic circuitry that is implemented in connection with processing and control circuits which are generally identified at 1012. Although not shown, computing system 1000 can include a system bus or data transfer system that couples the various components within the device. A system bus can include any one or combination of different bus structures, such as a memory bus or memory controller, a peripheral bus, a universal serial bus, and/or a processor or local bus that utilizes any of a variety of bus architectures.

Computing system 1000 also includes computer-readable media 1014, such as one or more memory devices that enable persistent and/or non-transitory data storage (i.e., in contrast to mere signal transmission), examples of which include random access memory (RAM), non-volatile memory (e.g., any one or more of a read-only memory (ROM), flash memory, EPROM, EEPROM, etc.), and a disk storage device. A disk storage device may be implemented as any type of magnetic or optical storage device, such as a hard disk drive, a recordable and/or rewriteable compact disc (CD), any type of a digital versatile disc (DVD), and the like. Computing system 1000 can also include a mass storage media device 1016.

Computer-readable media 1014 provides data storage mechanisms to store device data 1004, as well as various device applications 1018 and any other types of information and/or data related to operational aspects of computing system 1000. For example, an operating system 1020 can be maintained as a computer application with computer-readable media 1014 and executed on processors 1010. Device applications 1018 may include a device manager, such as any form of a control application, software application, signal-processing and control module, code that is native to a particular device, a hardware abstraction layer for a particular device, and so on.

Device applications 1018 also include any system components, engines, or managers to implement customizable health monitoring. In this example, device applications 1018 include manager 210 or sensor manager 320.

### CONCLUSION

Although embodiments of techniques using, and apparatuses including, customizable health monitoring have been described in language specific to features and/or methods, it is to be understood that the subject of the appended claims is not necessarily limited to the specific features or methods described. Rather, the specific features and methods are disclosed as example implementations of customizable health monitoring.

What is claimed is:

1. A computer-implemented method comprising:
receiving a health-monitoring prescription for a patient, the health-monitoring prescription requiring multiple health-monitoring acts, the multiple health-monitoring acts at prescribed times or events;
responsive to receiving the health-monitoring prescription and based on the prescribed times or events occurring, prompting the patient, for each of the prescribed times or events and through a mobile computing device, to initiate use of a health-monitoring device that is associated with the mobile computing device and capable of performing the multiple health-monitoring acts;
receiving, from the health-monitoring device, results from performance of the multiple health-monitoring acts for the patient; and
responsive to receiving the results from the performance of the health-monitoring acts, providing the results to an entity associated with the health-monitoring prescription.

2. The computer-implemented method as described in claim 1, wherein the health-monitoring prescription requires health-monitoring acts responsive to events and the events are sensed by the mobile computing device.

3. The computer-implemented method as described in claim 1, wherein the health-monitoring prescription requires health-monitoring acts responsive to events and the events include the patient eating, sleeping, exercising, or undergoing stress.

4. The computer-implemented method as described in claim 1, wherein the health-monitoring prescription requires health-monitoring acts responsive to events and the events include a passively sensed physical condition of the patient and receiving results causes the health-monitoring device to perform a health-monitoring act requiring action by the patient.

5. The computer-implemented method as described in claim 1, wherein the health-monitoring prescription is received from a medical professional caring for the patient.

6. The computer-implemented method as described in claim 1, further comprising, after prompting the patient, receiving an indication from the health-monitoring device that the patient has initiated use of the health-monitoring device and causing the health-monitoring device to perform the multiple health-monitoring acts responsive to receiving the indication.

7. The computer-implemented method as described in claim 1, wherein providing the results to the entity is responsive to a period of time elapsing or a number of monitoring acts being performed.

8. The computer-implemented method as described in claim 1, wherein the health-monitoring prescription includes a dynamic adjustment mechanism indicating, without further instructions from an entity associated with the health-monitoring prescription, a different health-monitoring act than that of the multiple health-monitoring acts or at a different time or event than those of the prescribed times or events, the dynamic adjustment responsive to a result outside of a threshold of the health-monitoring prescription.

9. The computer-implemented method as described in claim 1, wherein the health-monitoring prescription includes a dynamic warning mechanism indicating, without further instruction from an entity associated with the health-monitoring prescription, a result of the one or more health-monitoring acts being determined to indicate an acute health condition.

10. A health-monitoring device comprising:
a human-health-monitoring sensor;
a wired or wireless transceiver capable of receiving and transmitting information to a mobile computing device associated with a patient;
one or more computer processors; and
one or more computer-readable media having instructions stored thereon that, responsive to execution by the one or more computer processors, implements a sensor manager configured to:
receive, through the wired or wireless transceiver, from the mobile computing device, and following a health-monitoring prescription associated with the patient and the patient's medical professional, instructions regarding a health-monitoring act;
responsive to the instructions, prepare to perform the health-monitoring act;
cause the human health-monitoring sensor to perform the health-monitoring act; and
provide a result of the performance of the health-monitoring act to the mobile computing device.

11. The health-monitoring device as recited in claim 10, wherein receiving and providing are performed through an audio jack or serial bus port of the mobile computing device.

12. The health-monitoring device as recited in claim 10, wherein the health-monitoring act prescribed in the health-monitoring prescription includes a blood pressure, the instructions indicate to turn on an interface of the health-monitoring device, preparing turns on the interface, performing the health-monitoring act measure's the patient's blood pressure, recording the result records the patient's blood pressure, and providing the result provides the patient's blood pressure to the mobile computing device.

13. The health-monitoring device as recited in claim 10, wherein the health-monitoring act prescribed in the health-monitoring prescription includes a glucose content, the instructions indicate to turn on an interface of the health-monitoring device, preparing turns on the interface, performing the health-monitoring act measure's the patient's glucose content, and providing the result provides the patient's glucose content to the mobile computing device.

14. The health-monitoring device as recited in claim 10, wherein the sensor manager is further configured, responsive to provision of the result, to receive second instructions to perform a more-complex or more-invasive health-monitoring act and, responsive to reception of the second instructions, prepare for, perform, record a second result of, and provide the second result of the more-complex or more-invasive health-monitoring act.

15. A computer-implemented method comprising:
receiving a health-monitoring prescription for a patient, the health-monitoring prescription requiring multiple health-monitoring acts at prescribed patient conditions;
determining, through one or more passive sensors of a mobile computing device or patient-associated data of the mobile computing device, that the prescribed patient condition is occurring;
responsive to the determination, prompting the patient to initiate use of a health-monitoring device that is associated with the mobile computing device and is capable of performing the multiple health-monitoring acts; and
responsive to receiving a result from the performance of one of the health-monitoring acts, providing the result to an entity associated with the health-monitoring prescription.

16. The computer-implemented method of claim 15, wherein the prescribed patient condition includes at work, exercising, asleep, or at a particular location or is determined based on a calendar associated with the patient.

17. The computer-implemented method of claim 15, wherein the prescription further includes other health-monitoring acts for which the patient is not prompted, the other health-monitoring acts performed by another health-monitoring device, the other health-monitoring device being non-mobile or performing the other health-monitoring acts using a passive sensor.

18. The computer-implemented method of claim 15, wherein the prescribed patient condition is determined to be occurring based on sensor data from the one or more passive sensors, the one or more passive sensors including an accelerometer, a touch sensor of a display screen, or a radar sensor.

19. The computer-implemented method of claim 18, wherein determining that the prescribed patient condition is occurring is based on the sensor data indicating a heart rate, body temperature, skin temperature, or body movement of the patient.

20. The computer-implemented method of claim 15, further comprising determining, based on the result and the health-monitoring prescription, to perform a more-complex or more-invasive health-monitoring act and prompting the patient to initiate use of the health-monitoring device to perform the more-complex or more-invasive health-monitoring act and recording or providing a second result of the more-complex or more-invasive health-monitoring act.
